(19) United States
(12) Patent Application Publication
(10) Pub. No.: US 2008/0221370 A1
(43) Pub. Date: Sep. 11, 2008

(54) Process for Preparing Polyisobutyl-Substituted Cyclohexanols

(75) Inventors: Arno Lange, Bad Durkheim (DE); Helmut Mach, Heidelberg (DE); Hans Peter Rath, Grunstadt (DE); Darijo Mijolovic, Mannheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/913,723

(22) PCT Filed: May 5, 2006
(86) PCT No.: PCT/EP2006/004241

§ 371 Date: Nov. 6, 2007

(30) Foreign Application Priority Data
May 6, 2005 (DE) 10 2005 021 093.7

(51) Int. Cl. C07C 35/08

(52) U.S. Cl. 568/835

(57) ABSTRACT

The present invention relates to a process for preparing polyisobutyl-substituted cyclo-hexanols by hydrogenating polyisobutyl-substituted hydroxybenzenes. The invention further relates to the polyisobutyl-substituted cyclohexanols obtainable by this process and functionalization products thereof, and to their use for the surface modification of inorganic or organic material.

The present invention relates to a process for preparing polyisobutyl-substituted cyclohexanols by hydrogenating polyisobutyl-substituted hydroxybenzenes. The invention further relates to the polyisobutyl-substituted cyclohexanols obtainable by this process and functionalization products thereof, and to their use for the surface modification of inorganic or organic material.

Amphiphilic polyalkenyl derivates which possess an unpolar tail and a polar head are valuable products owing to their surface properties and their interface behavior and may be used, for example, as corrosion inhibitors, friction reducers, emulsifiers, dispersants, etc. Amphiphilic polyalkenyl derivates whose polar head group is hydroxy-functionalized are additionally valuable intermediates for the preparation of corresponding acrylates and polyurethanes.

The preparation of hydroxy-functionalized polyisobutenes is described, for example, in WO 03/020822. To this end, a reactive polyisobutene having terminal double bonds is either hydroborated and subsequently converted using alkaline hydrogen peroxide to the corresponding alcohol, or the polyisobutene is hydroformylated and the resulting oxo product is hydrogenated to the alcohol. However, disadvantages of these processes are that they are associated with relatively high synthetic complexity, that the preparation via the oxo process requires specific catalysts, and that, moreover, the reactions usually do not proceed to completion or are accompanied by side reactions.

It therefore appears to be more advantageous to prepare amphiphilic alcohols by hydrogenating a polyisobutyl-substituted phenol to the corresponding cyclohexanol.

Processes for preparing uniform, low molecular weight, alkyl-substituted cyclohexanols from the corresponding alkylphenols are known. For example, U.S. Pat. No. 2,026,668 describes the hydrogenation of phenols which are substituted by tertiary alkyl groups, for example of triisobutylphenol or tetraisobutylphenol, in the presence of a hydrogenation catalyst at elevated temperatures and pressures to the corresponding cyclohexanol.

GB 1,025,438 describes a process for preparing alkylcyclohexanols by hydrogenating alkylphenols in the presence of finely divided nickel on an inert support as a hydrogenation catalyst at elevated temperatures and pressures in a hydrogenated petroleum ether as a solvent. The alkylphenol used is, for example, 4-diisobutylphenol.

In these prior art hydrogenation processes, chemically uniform products, i.e. alkylphenols having a defined composition, are always used. In particular, no polymeric alkylphenols which differ in the chain length of the alkyl groups are used. Moreover, the alkyl groups in these reactants are relatively short-chain and comprise a maximum of four isobutene units. Owing to the relatively short chain lengths and in particular owing to the use of uniform reactants, the hydrogenation products obtained can be purified readily, for example by crystallization or, as described in GB 1,025,438, by fractional distillation.

In contrast, when polymeric alkylphenols which differ in the chain length of the alkyl groups are used as reactants, it is no longer possible to purify the resulting hydrogenation products, since they, for example, can no longer be crystallized. From a certain chain length of the alkyl group, moreover, purification by distillation, for example, is ruled out. For example, S. Koch, Thesis, 2000, Freie Universität Berlin, page 11 discloses that it is barely still possible to purify polymeric substances.

In the context of the present invention, polymeric alkylphenols are understood to be alkylphenols whose alkyl group derives from polyolefins. As a result of their preparation process, polyolefins generally do not have uniform chain length and therefore do not constitute chemically uniform products, since the individual polymer chains comprise a more or less different number of polymerized monomers. Phenols substituted by polymeric groups are therefore also not chemically uniform. It is self-evident that the reaction of such phenols on the phenol ring likewise leads to chemically nonuniform products.

Reactions with polymer group-containing substrates differ fundamentally from the reaction on low molecular weight, chemically uniform reactants. Thus, the polymer radical “dilutes” the reaction center, which can influence its reactivity. It is also frequently impossible to carry out the reaction in a solvent in the actual sense, since uneconomic space-time yields are otherwise achieved. However, the result of this is that an optimal solvent polarity which would promote the reaction cannot be established. In spite of this, reaction conditions have to be established in such a way that firstly the polymer chain is not affected, but the reactive center simultaneously reacts very substantially, since purifying operations in polar group-containing products, as mentioned above, generally do not lead to the desired success. The discovery of reaction conditions for the very substantial reaction, proceeding in the correct direction, of a polymer group-containing substrate is therefore a particular challenge. Differences between reactions of uniform reactants and polymer-derived substrates are described, for example, in Houben-Weyl, Methoden der Organischen Chemie, [Methods of organic chemistry], volume XIV/2, 4th edition, 1963, Thieme Verlag Stuttgart, page 646 ff.

When polymeric alkylphenols are used as reactants, i.e. mixtures in which the components have alkyl groups of different chain lengths and, if appropriate, also a different number of alkyl groups on the phenol ring, in order to obtain by hydrogenation industrially useful products, i.e. products which consist predominantly of alkylcyclohexanols, it is necessary that the hydrogenation reaction proceeds with maximum yield and minimum side reactions.

It was therefore an object of the present invention to provide a process for preparing polymeric alkylcyclohexanols from polymeric alkylphenols as reactants, in which the reactants are converted very substantially to the corresponding alkylcyclohexanols.

The object was achieved by a process for preparing polyisobutyl-substituted cyclohexanols of the formula (I),

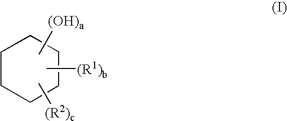

where
each R¹ is a polyisobutyl radical;
each R² is independently C₁-C₂₄-alkyl or C₁-C₂₄-alkoxy;
a and b are each independently from 1 to 3; and
c is from 0 to 4;
where the sum of a, b and c is from 2 to 6 and where each OH, R¹ and R² radical is bonded to different carbon atoms of the cyclohexane ring,
in which a polyisobutyl-substituted hydroxybenzene of the formula (II)

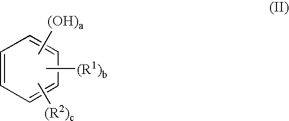

where R¹, R², a, b and c are each as defined above
is hydrogenated in the presence of a hydrogenation catalyst.

The reactant of the formula (II) is a polymer-derived substrate, i.e. the reactant is, as described above, a mixture of different hydroxybenzenes (II) which, as a result of the preparation process for the parent polyisobutene of the polyisobutyl radical R¹, differ in the chain length of the individual polyisobutyl groups R¹.

In addition to the chain length of the R¹ radical, the hydroxybenzenes (II) may also differ by the type of substituents R² and/or by the number a, b and/or c of the particular substituents OH, R¹ and R².

In the context of the present invention, C₁-C₄-alkyl is a linear or branched alkyl group having from 1 to 4 carbon atoms, such as methyl, ethyl, n-propyl, isopropyl, n-butyl, sec-butyl, isobutyl and tert-butyl. C₁-C₁₀-Alkyl is additionally, for example, pentyl, hexyl, heptyl, octyl, 2-ethylhexyl, nonyl or decyl and their positional isomers. C₁-C₂₀-Alkyl is additionally undecyl, dodecyl, tridecyl, tetradecyl, pentadecyl, hexadecyl, heptadecyl, octadecyl, nonadecyl or eicosyl and positional isomers thereof. C₁-C₂₄-Alkyl is additionally heneicosyl, docosyl, tricosyl or tetracosyl and positional isomers thereof. The alkyl radical is, if appropriate, substituted by at least one group which is selected from cycloalkyl, halogen, hydroxy, C₁-C₆-alkoxy, SR³ and NR³R⁴, where R³ and R⁴ are each independently H or C₁-C₄-alkyl. However, the alkyl radical is preferably unsubstituted.

C₁-C₄-Alkoxy is a C₁-C₄-alkyl radical which is bonded via an oxygen atom to the group bearing it. Examples thereof are methoxy, ethoxy, propoxy, isopropoxy, n-butoxy, 2-butoxy, isobutyloxy and tert-butyloxy. C₁-C₆-Alkoxy is additionally a C₅-C₆-alkyl radical which is bonded via an oxygen atom to the group bearing it. Examples thereof are pentoxy and hexoxy and positional isomers thereof. C₁-C₁₀-Alkoxy is additionally a C₇-C₁₀-alkyl radical which is bonded via an oxygen atom to the group bearing it. Examples thereof are heptoxy, octyloxy, nonyloxy and decyloxy and positional isomers thereof. C₁-C₂₄-Alkoxy is additionally a C₁₁-C₂₄-alkyl radical which is bonded via an oxygen atom to the group bearing it. Examples of C₁₁-C₂₄-alkyl radicals are those mentioned above.

The remarks which follow regarding suitable and preferred features of the process according to the invention and further subject matter of the invention apply both taken alone and in combination.

The polyisobutyl radical R¹ in the cyclohexanols of the formula (I) or in the hydroxybenzenes of the formula (II) preferably has a number-average molecular weight Mₙ of 150 to 30 000, more preferably from 200 to 20 000, even more preferably from 300 to 10 000 and in particular from 500 to 5000. The selection of the polyisobutyl radicals having particular molecular weights depends upon the application medium and intended application of the particular polyisobutyl-substituted cyclohexanols (I) and is determined in the individual case by those skilled in the art.

Furthermore, R¹ is preferably a radical which derives from what are known as “reactive” polyisobutenes which differ from the “low-reactivity” polyisobutenes by the content of terminal double bonds. Reactive polyisobutenes differ from low-reactivity polyisobutenes by comprising at least 50 mol %, based on the total number of polyisobutene macromolecules, of terminal double bonds. Particularly preferred R¹ radicals derive from reactive polyisobutenes having at least 60 mol % and in particular having at least 80 mol %, based on the total number of polyisobutene macromolecules, of terminal double bonds. The terminal double bonds may either be vinyl double bonds [—CH═C(CH₃)₂] (β-olefin) or vinylidene double bonds [—CH—C(═CH₂)—CH₃] (α-olefin). They are preferably vinylidene double bonds.

Moreover, the R¹ radical preferably derives from those polyisobutenes which have uniform polymer skeletons. Uniform polymer skeletons are those which are formed essentially from only one monomer (here: isobutene). Uniform polymer skeletons are possessed in particular by those polyisobutenes which are formed to an extent of at least 85% by weight, preferably to an extent of at least 90% by weight and more preferably to an extent of at least 95% by weight, of isobutene units.

Furthermore, the polyisobutyl radical derives from polyisobutenes having a polydispersity index (PDI) of preferably from 1.05 to 10. Polydispersity is understood to be the quotient of weight-average molecular weight M_{w} and number-average molecular weight Mₙ (PDI=M_{w}/Mₙ). The selection of polyisobutyl radicals having a particular PDI is determined by the intended use of the polyisobutyl-substituted cyclohexanol (I) and is selected correspondingly by those skilled in the art. In general, the PDI value of a compound or of a radical for a given Mₙ correlates with its viscosity. Accordingly, for applications in which low miscibility or processibility with the application medium and thus low viscosity is required, a polyisobutyl radical having a PDI of preferably <3.0 is selected. For surface modifications in the form of coatings, on the other hand, a higher viscosity is frequently desired, so that in this case, polyisobutyl radicals having a PDI in the range from 1.5 to 10 are preferred. Polyisobutyl-substituted cyclohexanols (I) having a narrow molecular weight distribution (PDI from about 1.05 to about 2.0) of the polyisobutyl radical are suitable, for example, for use as a detergent or dispersant in fuel and lubricant compositions, as an additive in printing systems, in polymers or in monolayers for hydrophobization. Polyisobutyl radicals having an average molecular weight distribution (PDI from about 1.6 to about 2.5) are suitable, for example, for use of the polyisobutyl-substituted cyclohexanol (I) in emulsions or dispersions and for hydrophobizing basic materials such as calcium carbonate (for example in the form of mortar), gypsum or cement, while those having a broad molecular weight distribution (PDI from about 2.1 to about 10) are suitable for use as corrosion inhibitors or likewise for hydrophobizing basic materials. When the polyisobutyl-substituted cyclohexanols (I) prepared in accordance with the invention are to be used especially as dispersants in fuel and lubricant compositions, R¹ derives from polyisobutenes having a PDI of preferably ≦3.0, more preferably ≦1.9, in particular ≦1.7 and especially ≦1.5.

In a particularly preferred embodiment of the invention, R¹ derives from polyisobutenes which are obtainable by living cationic polymerization. Living cationic polymerization refers generally to the polymerization of isoolefins or vinyl aromatics in the presence of metal halides or semimetal halides as Lewis acid catalysts, and tert-alkyl halides, benzyl or allyl halides, benzyl or allyl esters, or benzyl or allyl ethers as initiators, which form a carbocation or a cationogenic complex with the Lewis acid. A comprehensive review on this subject can be found in Kennedy/Ivan “Carbocationic Macromolecular Engineering”, Hanser Publishers 1992.

In an alternatively particularly preferred embodiment of the invention, R¹ derives from telechelic polyisobutenes which are obtainable by living cationic polymerization. Telechelic polyisobutenes are understood to be polymers which have two or more reactive end groups. These end groups are in particular carbon-carbon double bonds which can be further functionalized, halogen atoms, initiator molecules incorporated into the polymer which themselves have a functional group, for example a carbon-carbon double bond, or groups functionalized with a terminating agent. To prepare telechelic polyisobutenes, a bifunctional initiator such as dicumyl chloride is generally used.

Such telechelic polyisobutenes are described, for example, in EP-A-722957, WO 02/48215, WO 03/074577 or in the German patent application 10328854.6, which are hereby fully incorporated by reference.

Polyisobutyl radicals R¹ which derive from telechelic polyisobutenes have, in compounds (II), preferably at least one further hydroxybenzene group (II.a)

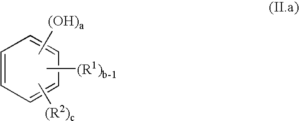

in which R¹, R², a, b and c are each as defined above.

For example, at least one of the R¹ in compounds (II) is substituted by 1, 2 or 3, preferably 1 or 2, further hydroxybenzene groups (II.a). R¹ is more preferably substituted by one further hydroxybenzene group (II.a). The maximum possible number of (II.a) radicals which may bear an R¹ radical depends on the number of reactive end groups which are present in the parent polyisobutene molecule of the R¹ radical. For instance, an R¹ radical which derives from a bifunctional polyisobutene (i.e. a polyisobutene having two reactive end groups) may bear a maximum of one additional (II.a) group, while an R¹ radical which derives from a trifunctional polyisobutene (i.e. a polyisobutene having three reactive end groups) may bear a maximum of two additional (II.a) groups.

Consequently, polyisobutyl radicals R¹ which derive from telechelic polyisobutenes have, in compounds (I), preferably at least one further cyclohexanol group (I.a)

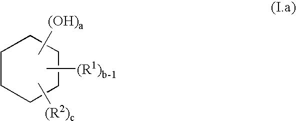

in which R¹, R², a, b and c are each as defined above.

For example, at least one of the R¹ radicals in compounds (I) is substituted by 1, 2 or 3, preferably 1 or 2, further cycloalkanol groups (I.a). R¹ is more preferably substituted by one further cycloalkanol group (I.a). With regard to the maximum possible number of (I.a) groups which may bear an R¹ radical, the same applies as was stated above.

Polyisobutyl-substituted hydroxybenzenes (II) in which the R¹ radical has at least one further hydroxybenzene group (II.a) are obtainable, for example, by using at least one bifunctional polyisobutene, for example a polyisobutene which comprises halogen atoms or carbon-carbon double bonds on at least two chain ends, in the reaction described below for the alkylation of hydroxybenzenes.

Polyisobutyl-substituted cyclohexanols (I), in which the R¹ radical has at least one further cyclohexanol group (I.a) are obtainable, for example, by hydrogenating hydroxybenzenes (II) in which the R¹ radical has at least one further hydroxybenzene group (II.a) by the process according to the invention.

In a preferred embodiment, the R¹ radical bears no or only one further group (I.a) or (II.a). Specifically, the R¹ radical does not bear any (I.a) or (II.a) group.

More preferably, a and b in the polyisobutyl-substituted cyclohexanols (I) and hydroxybenzenes (II) are each 1. In particular, the R¹ radical is arranged in the p-position relative to the hydroxyl group.

The R² radical is preferably C₁-C₁₀-alkyl, more preferably C₁-C₆-alkyl, in particular C₁-C₄-alkyl and especially methyl.

In compounds (I) and (II), c is preferably 0.

Polyisobutyl-substituted aromatic hydroxyl compounds of the formula (II) and their preparation are known, for example, from GB-A-1159368, U.S. Pat. No. 4,429,099, WO 94/14739, from J. Polym. Sci. A, 31, 1938 (1993), from WO 02/26840, and from Kennedy, Guhaniyogi and Percec, Polym. Bull. 8, 563 (1970), which are hereby fully incorporated by reference.

For example, the polyisobutyl-substituted aromatic hydroxy compound of the formula (II) is obtainable, for example, by the reaction (alkylation) of an aromatic hydroxyl compound substituted by c R² radicals with a polyisobutene.

Aromatic hydroxyl compounds preferred for the alkylation are unsubstituted or mono- or disubstituted phenol, and also unsubstituted and mono- or disubstituted di- and trihydroxybenzenes. In the di- and trihydroxyl compounds, the hydroxyl groups are preferably not in the o-position relative to one another. Particular preference is given to using phenols. Suitable substituted phenols are in particular mono-ortho-substituted phenols. Preferred substituents are C₁-C₄-alkyl groups, in particular methyl and ethyl. Particularly preferred for the alkylation with polyisobutenes are unsubstituted phenol and 2-methylphenol. However, also suitable are optionally substituted di- and trihydroxybenzenes.

The polyisobutene used in the alkylation reaction may be any common and commercially available polyisobutene.

In the context of the present invention, the term “polyisobutene” also includes oligomeric isobutenes such as dimeric, trimeric or tetrameric isobutene.

In the context of the present invention, polyisobutenes are also understood to be all polymers obtainable by cationic polymerization which preferably comprise at least 60% by weight of isobutene, more preferably at least 80% by weight, even more preferably at least 90% by weight and in particular at least 95% by weight, of isobutene in polymerized form. In addition, the polyisobutenes may comprise, in copolymerized form, further butene isomers, such as 1- or 2-butene, and also different olefinically unsaturated monomers which are copolymerizable with isobutene under cationic polymerization conditions.

Suitable isobutene feedstocks for the preparation of polyisobutenes which are suitable as reactants for the process according to the invention are accordingly both isobutene itself and isobutenic C₄ hydrocarbon streams, for example C₄ raffinates, C₄ cuts from isobutene dehydrogenation, C₄ cuts from steamcrackers, FCC-crackers (FCC: fluid catalyzed cracking), provided that they have substantially been freed of 1,3-butadiene comprised therein. Particularly suitable C₄ hydrocarbon streams comprise generally less than 500 ppm, preferably less than 200 ppm, of butadiene. When C₄ cuts are used as the starting material, the hydrocarbons other than isobutene assume the role of an inert solvent.

Useful copolymerizable monomers include vinylaromatics such as styrene and α-methylstyrene, C₁-C₄-alkylstyrenes such as 2-, 3- and 4-methylstyrene, and also 4-tert-butylstyrene, isoolefins having from 5 to 10 carbon atoms such as 2-methylbutene-1,2-methylpentene-1,2-methylhexene-1,2-ethylpentene-1,2-ethylhexene-1 and 2-propylheptene-1. Further useful comonomers include olefins which have a silyl group such as 1-trimethoxysilylethene, 1-(trimethoxysilyl)propene, 1-(trimethoxysilyl)-2-methylpropene-2,1-[tri(methoxyethoxy)silyl]ethene, 1-[tri(methoxyethoxy)silyl]propene, and 1-[tri(methoxyethoxy)silyl]-2-methylpropene-2.

Suitable polyisobutenes are all polyisobutenes obtainable by conventional cationic or living cationic polymerization. However, preference is given to what are known as “reactive” polyisobutenes and also to telechelic polyisobutenes which have already been described above.

Suitable polyisobutenes are, for example, the Glissopal brands from BASF AG, for example Glissopal 550, Glissopal 1000 and Glissopal 2300, and the Oppanol brands from BASF AG, such as Oppanol B10, B12 and B15.

Processes for preparing suitable polyisobutenes are known, for example from DE-A 27 02 604, EP-A 145 235, EP-A 481 297, EP-A 671 419, EP-A 628 575, EP-A 807 641 and WO 99/31151. Polyisobutenes which are prepared by living cationic polymerization of isobutene or isobutene-containing monomer mixtures are described, for example, in U.S. Pat. No. 4,946,899, U.S. Pat. No. 4,327,201, U.S. Pat. No. 5,169,914, EP-A 206 756, EP-A 265 053, WO 02/48216 and in J. P. Kennedy, B. Ivan, “Designed Polymers by Carbocationic Macromolecular Engineering”, Oxford University Press, New York 1991. Publications on processes for preparing telechelic isobutenes have already been described above. These and other publications which describe polyisobutenes are hereby incorporated fully by reference.

Depending on the polymerization process, the polydispersity index (PDI=M_{w}/Mₙ) of the resulting polyisobutenes is from about 1.05 to 10. Polymers from living cationic polymerization generally have a PDI of from about 1.05 to 2.0. The molecular weight distribution of the polyisobutenes used in the process according to the invention has a direct effect on the molecular weight distribution of the cyclohexanol (I) to be prepared. As already detailed, depending on the intended use of the cyclohexanol (I), polyisobutenes having a narrow, an average or a broad molecular weight distribution are selected.

The alkylation to the polyisobutyl-substituted dihydroxybenzene (II) is effected preferably in the presence of a suitable catalyst. Suitable alkylation catalysts are, for example, protonic acids such as sulfuric acid, phosphoric acid and organic sulfonic acids, e.g. trifluoromethansulfonic acid, Lewis acids such as aluminum trihalides, e.g. aluminium trichloride or aluminium tribromide, boron trihalides, e.g. boron trifluoride and boron trichloride, tin halides, e.g. tin tetrachloride, titanium halides, e.g. titanium tetrabromide and titanium tetrachloride; and iron halides, e.g. iron trichloride and iron tribromide. The Lewis acids are, if appropriate, used together with Lewis bases such as alcohols, in particular C₁-C₆-alkanols, phenols or aliphatic or aromatic ethers, for example diethyl ether, diisopropyl ether or anisol. Preference is given to adducts of boron trihalides, in particular boron trifluoride, in combination with the aforementioned Lewis bases. Particular preference is given to boron trifluoride etherate and boron trifluoride phenolate. For practical reasons, particularly the latter is suitable, since it is formed when boron trifluoride is introduced into the phenol-containing reaction mixture.

The alkylation product may subsequently be used crude or preferably purified in the process according to the invention. For purification, the reaction mixture may be freed of excess phenol and/or catalyst, for example, by extraction with solvents, preferably polar solvents such as water or C₁-C₆-alkanols or mixtures thereof, by stripping, i.e. by passing steam through or, if appropriate, heating gases, for example nitrogen, by distillation or by means of basic ion exchangers, as described in the German patent application P 10060902.3.

In the process according to the invention, the hydrogenation catalysts may generally be all prior art catalysts which catalyze the hydrogenation of aromatics to the corresponding cycloalkanes, and more specifically of hydroxyaromatics to the corresponding hydroxycycloalkanes. The catalysts may be used either in heterogeneous phase or as homogeneous catalysts. The hydrogenation catalysts preferably comprise at least one metal of group VIII.

Particularly suitable metals of group VII are selected from ruthenium, cobalt, rhodium, nickel, palladium und platinum.

The metals may also be used in the form of mixtures. Moreover, the catalysts may comprise, in addition to the metals of group VII, also small amounts of further metals, for example metals of group VIIa, in particular rhenium, or metals of group Ib, i.e. copper, silver or gold. Particularly preferred metals of group VII are ruthenium, nickel, palladium and platinum, in particular ruthenium, nickel and palladium, and more preferably ruthenium and nickel. The catalyst especially comprises nickel as the catalytically active species.

When a heterogeneous catalyst is used, it is suitably present in finely divided form. The finely divided form is achieved, for example, as follows:
a) Black catalyst: shortly before use as a catalyst, the metal is deposited reductively from the solution of one of its salts.
b) Adams catalyst: the metal oxides, in particular the oxides of platinum and palladium, are reduced in situ by the hydrogen used for the hydrogenation.
c) Skeletal or Raney catalyst: the catalyst is prepared as a “metal sponge” from a binary alloy of the metal (in particular nickel or cobalt) with aluminum or silicon by leaching out one partner with acid or alkali. Residues of the original alloy partner often act synergistically.
d) Supported catalyst: black catalysts can also be precipitated on the surface of a support substance. Suitable supports and support materials are described below.

Such heterogeneous catalysts are described in general form, for example, in Organikum, 17th edition, VEB Deutscher Verlag der Wissenschaften, Berlin, 1988, p. 288. Moreover, heterogeneous hydrogenation catalysts which are suitable for the reduction of aromatics to cycloalkanes are described in detail in the following documents:

U.S. Pat. No. 3,597,489, U.S. Pat. No. 2,898,387 and GB 799,396 describe the hydrogenation of benzene to cyclohexane over nickel and platinum catalysts in the gas or liquid phase. GB 1,155,539 describes the use of a rhenium-doped nickel catalyst for the hydrogenation of benzene. U.S. Pat. No. 3,202,723 describes the hydrogenation of benzene with Raney nickel. Ruthenium-containing suspension catalysts which are doped with palladium, platinum or rhodium are used in SU 319582 for the hydrogenation of benzene to cyclohexane. Alumina-supported catalysts are described in U.S. Pat. No. 3,917,540 and U.S. Pat. No. 3,244,644. The hydrogenation catalysts described in these documents are incorporated fully by reference.

Depending on the configuration of the hydrogenation process, the support material can take various forms. When the hydrogenation is carried out in liquid phase mode, the support material is generally used in the form of a fine powder. On the other hand, when the catalyst is used in the form of a fixed bed catalyst, the support material used is, for example, shaped bodies. Such shaped bodies may be present in the form of spheres, tablets, cylinders, hollow cylinders, Raschig rings, extrudates, saddles, stars, spirals, etc., having a size (length of longest dimension) of from about 1 to 30 mm. Moreover, the supports may be present in the form of monoliths, as described, for example, in DE-A-19642770. In addition, the supports may be used in the form of wires, sheets, grids, meshes, fabrics and the like.

The supports may consist of metallic or nonmetallic, porous or nonporous material.

Suitable metallic materials are, for example, highly alloyed stainless steels. Suitable nonmetallic materials are, for example, mineral materials, for example natural and synthetic minerals, glasses or ceramics, plastics, for example synthetic or natural polymers, or a combination of the two.

Preferred support materials are carbon, in particular activated carbon, silicon dioxide, in particular amorphous silicon dioxide, alumina, and also the sulfates and carbonates of the alkaline earth metals, calcium carbonate, calcium sulfate, magnesium carbonate, magnesium sulfate, barium carbonate and barium sulfate.

The catalyst may be applied to the support by customary processes, for example by impregnating, wetting or spraying the support with a solution which comprises the catalyst or a suitable precursor thereof.

Suitable supports and processes for applying the catalyst thereto are described, for example, in DE-A-10128242, which is hereby fully incorporated by reference.

It is also possible to use homogeneous hydrogenation catalysts in the process according to the invention. Examples thereof are the nickel catalysts which are described in EP-A-0668257. However, disadvantages of use of homogeneous catalysts are their preparation costs and also the fact that they generally cannot be regenerated.

Therefore, preference is given to using heterogeneous hydrogenation catalysts in the process according to the invention.

The heterogeneous catalysts used in the process according to the invention more preferably comprise at least one metal of transition group VII which is selected from ruthenium, nickel, cobalt, palladium and platinum, and which has, if appropriate, been doped with a further transition metal, in particular with one of transition group VIIa, Ib or IIb and in particular with rhenium.

The metal is more preferably used in supported form or as metal sponge. Examples of supported catalysts are in particular palladium, nickel or ruthenium on carbon, in particular activated carbon, silicon dioxide, in particular on amorphous silicon dioxide, barium carbonate, calcium carbonate, magnesium carbonate or alumina, and the supports may be present in the above-described shapes. Preferred support shapes are the above-described shaped bodies.

The metallic catalysts may also be used in the form of their oxides, in particular palladium oxide, platinum oxide or nickel oxide, which are then reduced under the hydrogenation conditions to the corresponding metals.

The metal sponge used is in particular Raney nickel.

The hydrogenation catalyst used in the process according to the invention is especially Raney nickel.

The amount of catalyst to be used depends on factors including the particular catalytically active metal and its use form, and may be determined in the individual case by those skilled in the art. For example, a nickel- or cobalt-containing hydrogenation catalyst is used in an amount of preferably from 0.5 to 70% by weight, more preferably from 1 to 20% by weight and in particular from 2 to 10% by weight, based on the weight of the polyisobutyl-substituted hydroxybenzene (II) used. The amount of catalyst specified relates to the amount of active metal, i.e. to the catalytically active component of the catalyst. When noble metal catalysts are used which comprise, for example, platinum or palladium, values smaller by a factor of 10 apply.

The hydrogenation is effected at a temperature of preferably from 20 to 250° C., more preferably from 50 to 240° C. and in particular from 150 to 220° C.

The reaction pressure of the hydrogenation reaction is preferably in the range from 1 to 300 bar, more preferably from 50 to 250 bar and in particular from 150 to 230 bar.

Both reaction pressure and reaction temperature depend upon factors including the activity and amount of the hydrogenation catalyst used and may be determined in the individual case by those skilled in the art.

In a preferred embodiment (a) of the process according to the invention, the polyisobutyl-substituted hydroxybenzene (II) to be hydrogenated is at least partly deprotonated on at least one hydroxyl group. The deprotonation may be effected either before the actual hydrogenation reaction or during the hydrogenation. However, preference is given to effecting the at least partial deprotonation before the hydrogenation reaction.

Suitable for the deprotonation are all common bases which can convert a phenol to the phenoxide. These include inorganic bases such as alkali metal and alkaline earth metal hydroxides, e.g. sodium hydroxide, potassium hydroxide, magnesium hydroxide and calcium hydroxide, alkali metal carbonates, e.g. sodium carbonate and potassium carbonate, alkali metal and alkaline earth metal oxides such as sodium oxide, lithium oxide, calcium oxide and magnesium oxide, and also alkali metal and alkaline earth metal hydrides such as sodium hydride or calcium hydride. However, also suitable are organic bases, for example alkoxides such as sodium methoxide and potassium tert-butoxide. However, preference is given to using inorganic bases such as those mentioned above, more preferably alkali metal or alkaline earth metal hydrides and especially sodium hydride.

The deprotonation of the reactant has the effect that the hydrogenation proceeds with a distinctly better conversion than in an nonalkaline medium. In this context, it is sufficient that only a portion of the hydroxybenzene used is deprotonated.

The base used for the deprotonation is preferably used in such an amount that at least 0.1 mol %, for example from 0.1 to 50 mol % or preferably from 0.1 to 30 mol %, more preferably at least 1 mol %, for example from 1 to 20 mol %, and in particular at least 2 mol %, for example from 2 to 20 mol %, of the hydroxyl groups comprised in the polyisobutyl-substituted hydroxybenzene (II) are deprotonated.

In an alternative, preferred embodiment (b) of the process according to the invention, the hydroxybenzene (II) used is repeatedly hydrogenated. In this embodiment, as soon as no further hydrogen consumption can be detected, further hydrogen is injected. Before the injection of hydrogen, preference is given to first adding fresh catalyst.

The repeated hydrogenation (“post-hydrogenation”) can be effected either alternatively or additionally to the (partial) deprotonation of the reactant.

Particular preference is given to the embodiment (a) in which the reactant is at least partly deprotonated. However, it is additionally also possible in this embodiment to post-hydrogenate the resulting reaction product according to embodiment (b).

Both the deprotonation in the preferred embodiment (a) and the actual hydrogenation are effected preferably in a suitable solvent. Suitable solvents are those which are inert under the reaction conditions, i.e. neither react with the reactant or product nor are changed themselves. In particular, suitable solvents are not themselves hydrogenated under the hydrogenation conditions. The suitable solvents include alkanes, in particular C₅-C₁₀-alkanes such as pentane, hexane, heptane, octane, nonane, decane and isomers thereof, cycloalkanes, in particular C₅-C₈-cycloalkanes such as cyclopentane, cyclohexane, cycloheptane or cyclooctane, open-chain and cyclic ethers such as diethyl ether, methyl tert-butyl ether, tetrahydrofuran or 1,4-dioxane, and alcohols, in particular C₁-C₃-alkanols such as methanol, ethanol, n-propanol or isopropanol. Also suitable are mixtures of the aforementioned solvents. Preferred solvents are C₅-C₁₀ alkanes and mixtures thereof, particular preference being given to C₅-C₈-alkanes such as pentane, hexane, heptane and octane and positional isomers thereof. Preference is also given to the use of mixtures of such C₅-C₈-alkanes. Suitable alkane mixtures are, for example, petroleum ethers. Petroleum ethers are low-boiling benzine fractions (boiling point from about 25 to 80° C.) which consist mainly of hydrocarbons, in particular of alkanes and cycloalkanes. However, greater preference is given to using as solvents alkanes, in particular C₅-C₇-alkanes such as pentane, hexane or heptane and mixtures of these alkanes. Especially heptane is used.

The hydrogen required for the hydrogenation may be used either in pure form or in the form of hydrogen-containing gas mixtures. However, the latter must not comprise any damaging amounts of catalyst poisons such as CO. Examples of suitable hydrogen-containing gas mixtures are those from the reforming process. However, preference is given to using hydrogen in pure form.

The process according to the invention may be configured either continuously or batchwise.

The hydrogenation is generally carried out in such a way that the polyisobutyl-substituted hydroxybenzene (II) is initially charged in the solvent. This reaction solution is subsequently preferably initially admixed with the hydrogenation catalyst before the introduction of hydrogen then begins. Depending on the hydrogenation catalyst used, the hydrogenation is effected at elevated temperature and/or at elevated pressure. For the reaction under pressure, the customary pressure vessels known from the prior art, such as autoclaves, stirred autoclaves and pressure reactors, may be used. When elevated hydrogen pressure is not employed, useful apparatus is the customary prior art reaction apparatus which is suitable for standard pressure. Examples thereof are customary stirred tanks which are preferably equipped with evaporated cooling, suitable mixers, introduction devices, if appropriate heat exchanger elements and inertization devices. In the case of continuous reaction, the hydrogenation may be carried out under standard pressure in reaction vessels, stirred reactors, fixed bed reactors and the like which are customary for this purpose.

In the preferred embodiment (a) of the process according to the invention, the solution of polyisobutyl-substituted hydroxybenzene (II) in the solvent is admixed with the base. Alternatively, the base intended for deprotonation is initially charged in the solvent and this solution or suspension is admixed with the hydroxybenzene (II) to be hydrogenated, although the first procedure is preferred. Depending on the base used, the deprotonation which then sets in proceeds exothermically and, for example, when metal hydrides are used as the base, is also accompanied by gas evolution (hydrogen). When the deprotonation is carried out in a reaction vessel other than the hydrogenation apparatus, preference is given in this case to initially waiting for gas evolution and/or heat evolution and only then feeding the reaction solution to the hydrogenation vessel. However, when the deprotonation takes place in the hydrogenation vessel, it is not absolutely necessary to wait for abatement of heat evolution and/or of gas formation. This reaction solution is subsequently preferably initially admixed with the hydrogenation catalyst before the introduction of hydrogen then begins. Alternatively, the catalyst may also be already present during the deprotonation operation, although the former method is preferred. Alternatively, the hydroxybenzene to be hydrogenated may also be deprotonated during the actual hydrogenation operation. For this purpose, the base intended for the deprotonation is added shortly before, shortly after or simultaneously with the addition of the catalyst, or even not until during the introduction of the hydrogen. The latter may of course only be done when the hydrogenation reaction is carried out under standard pressure. However, preference is given to effecting the deprotonation before the actual hydrogenation reaction.

In the alternative preferred embodiment (b), no base is used. Instead, single or multiple post-hydrogenation is effected. This variant is suitable in particular for the performance of the hydrogenation under pressure. The post-hydrogenation is preferably effected in such a way that the procedure is initially as in variant (a), but without addition of base. After the customary reaction time, either the hydrogen pressure is increased once more as soon as the hydrogen pressure no longer changes, or the reaction vessel is preferably first decompressed and then, if appropriate after addition of fresh catalyst, charged again with hydrogen up to the desired pressure. This operation can be repeated more than once.

It is self-evident that this multiple post-hydrogenation may also be carried out in the preferred embodiment (a).

On completion of hydrogenation, the catalyst and the solvent are generally removed. The heterogeneous catalyst is preferably removed by filtration or by sedimentation and removal of the upper, product-containing phase. Other removal processes for removing solids from solutions, for example centrifugation, are also suitable for removing the heterogeneous catalyst. Homogeneous catalysts are removed by customary processes for separating single-phase mixtures, for example by chromatographic methods. If appropriate, it may be necessary, depending on the catalyst type, to deactivate it before the removal. This can be effected by customary processes, for example by washing the reaction solution with protic solvents, for example with water or with C₁-C₃-alkanols such as methanol, ethanol, propanol or isopropanol, which may be basified or acidified if required.

The solvent is removed by customary processes, for example by distillation, in particular under reduced pressure.

In a preferred embodiment, the process according to the invention is carried out according to the preferred embodiment (a).

The process according to the invention affords polyisobutyl-substituted cyclohexanols (I) in high yields and high purity. Preference is given to conducting the process according to the invention to a yield of polyisobutyl-substituted cyclohexanols of at least 75%, more preferably at least 80%, even more preferably at least 85%, in particular at least 90% and especially at least 95% of theory. In particular, the reaction product prepared by the process according to the invention preferably comprises less than 5% by weight, more preferably less than 2% by weight and in particular less than 1% by weight, based on the total weight of the reaction mixture obtained after the removal of catalyst and solvent, of polyisobutyl-substituted cyclohexanes, i.e. hydrogenation products in which the hydroxyl group has also been reduced.

It will be appreciated that the product prepared by the process according to the invention is a mixture of different polyisobutyl-substituted cyclohexanols (I) with different R¹ radicals which differ in particular in the number-average molecular weight Mₙ. The different number-average molecular weight of the R¹ radicals results, for example, from a different number of copolymerized isobutene molecules or else from the particular R¹ radicals bearing a different number of groups (I.a). In addition, the cyclohexanols (I) may differ by the type of the R² radical and/or by the number a, b and/or c of the particular substituents OH, R¹ and R².

The polyisobutyl-substituted cyclohexanols (I) obtained by the process according to the invention may be present either in the form of pure geometric isomers or as a mixture of different geometric isomers. Especially in the case that a and b in formula (I) are each 1 and c is 0, the reaction product may be obtained as substantially pure cis isomer, substantially pure trans isomer or as a mixture of cis and trans isomers.

Owing to the good yields and high purities, the hydrogenation products may be sent directly to their use without further purification or subjected to further functionalization reactions.

The invention further relates to a composition comprising polyisobutyl-substituted cyclohexanols of the formula (I)

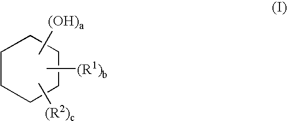

in which R¹, R², a, b and c are each as defined above
which is obtainable by the process according to the invention.

The inventive composition is a mixture of different cyclohexanols I which differ by the R¹ radicals and optionally additionally by the R² radicals and/or the number a, b and/or c of the OH, R¹ and R² radicals. The R¹ radicals differ in the chain length and if appropriate also in the type and number of any additional (I.a) group(s) present.

Preferred embodiments of these variables and of the process according to the invention have likewise been described above.

In particular, the R¹ radicals of the cyclohexanols (I) comprised in preferred compositions have a number-average molecular weight Mₙ of from 150 to 30 000, more preferably from 200 to 20 000, even more preferably from 300 to 10 000 and in particular from 500 to 5000.

Since polymer-derived reactants (I) whose polyisobutyl radical is nonuniform are used in the process according to the invention, the inventive composition comprises various polyisobutyl-substituted cyclohexanols of the formula (I) which differ by the number-average molecular weight Mₙ of the R¹ radical. In addition, the cyclohexanols may differ by the type of R² radical and/or by the number a, b and/or c of the particular substituents OH, R¹ and R².

The invention further relates to functionalization products of the polyisobutyl-substituted cyclohexanols of the formula (I), obtainable by reacting the polyisobutyl-substituted cyclohexanols (I)
(a) with an olefinically unsaturated mono- or dicarboxylic acid or a derivative thereof and, if appropriate, subsequently polymerizing the olefinically unsaturated product formed, or with the polymer of an olefinically unsaturated mono- or dicarboxylic acid or a derivative thereof;
(b) with an allyl halide and, if appropriate, subsequently polymerizing the allyl ether formed;
(c) with an alkylene oxide;
(d) with an isocyanate, diisocyanate or triisocyanate;
(e) with a carbonic acid derivative or with saturated or aromatic dicarboxylic acids or derivatives thereof; or
(f) with ammonia or amines NHR^{a}R^{b}, where R^{a} is C₁-C₂₄-alkyl and R^{b} is H or C₁-C₂₄-alkyl, and, if appropriate, further reaction of the amine formed
   (f.1) with at least one olefinically unsaturated mono- or dicarboxylic acid or a derivative thereof and, if appropriate, subsequently polymerizing the olefinically unsaturated product formed, or with the polymer of an olefinically unsaturated mono- or dicarboxylic acid or a derivative thereof;
   (f.2) with an alkylene oxide; or
   (f.3) with an isocyanate, diisocyanate or triisocyanate; or
   (f.4) with a carbonic acid derivative or with saturated or aromatic dicarboxylic acids or derivatives thereof.
(a) Reaction of polyisobutyl-substituted cyclohexanols (I) with an olefinically unsaturated mono- or dicarboxylic acid or a derivative thereof and, if appropriate, subsequently polymerizing the olefinically unsaturated product formed, or with the polymer of an olefinically unsaturated mono- or dicarboxylic acid or a derivative thereof

The olefinically unsaturated mono- or dicarboxylic acids are preferably α,β-unsaturated mono- or dicarboxylic acids such as acrylic acid, methacrylic acid, itaconic acid, citraconic acid, maleic acid or fumaric acid. Suitable derivatives of these mono- or dicarboxylic acids are those which can be condensed with the cyclohexanol (I) to give polyisobutyl-substituted cyclohexane esters of these mono- or dicarboxylic acids. Examples thereof are the halides, the mixed or symmetrical anhydrides, and esters, in particular the C₁-C₄-alkyl esters, of these mono- or dicarboxylic acids. The esterification, both of the monomeric and of the polymeric mono- or dicarboxylic acids, is effected by customary prior art processes, for example as described in Jerry March, Advanced Organic Chemistry, 3rd edition, John Wiley & Sons, p. 348 ff.

The monomeric mono- or dicarboxylic acids esterified with the inventive polyisobutyl-substituted cyclohexanol (I) may likewise be polymerized by known prior art processes under reaction conditions as are customary for the polymerization of olefinically unsaturated mono- or dicarboxylic acids, for example of acrylic acids or acrylates, and as are described, for example, in EP-A-0839839 and in the literature cited therein, which are fully incorporated by reference.

The polymer of an olefinically unsaturated mono- or dicarboxylic acid or derivative thereof may be either the homopolymer or the copolymer of this carboxylic acid or derivative thereof with suitable comonomers. Suitable comonomers are those which are copolymerizable with olefinically unsaturated mono- or dicarboxylic acids under the polymerization conditions customary therefor. Examples thereof are olefins such as ethylene, propylene, butylene and the like, dienes such as 1,3-butadiene, vinylaromatics such as styrene or α-methylstyrene, vinyl esters such as vinyl acetate, vinyl ethers and the like. Here too, suitable derivatives of the polymerized mono- or dicarboxylic acids are those which, as described above, are condensable with the cyclohexanol (I) to give esters.
(b) Reaction with an allyl halide and, if appropriate, subsequent polymerization of the allyl ether formed

The inventive cyclohexanol (I) is reacted with an allyl halide under reaction conditions as are customary for etherifications and as are described, for example, in Organikum, VEB Deutscher Verlag der Wissenschaften, 17th edition, Berlin, p. 196 ff.

The allyl ether formed may be polymerized if desired. Suitable polymerization conditions are known from the prior art.
(c) Reaction with an alkylene oxide

Suitable alkylene oxides are in particular ethylene oxide and 1,2-propylene oxide. These are nucleophilically attacked by the hydroxyl group of the cyclohexanol (I) obtained in accordance with the invention and react to give polyalkoxylates, i.e. to give polyether moieties having repeat units of the formula AOₙ-A-OH, in which A is, for example, 1,2-ethylene or 1,2-propylene, and n is, for example, from 1 to 100. Suitable alkoxylation conditions are known from the prior art and are described, for example, in EP-A-0277345 or WO 02/00599 and in the literature cited therein, which are fully incorporated by reference.
(d) Reaction with an isocyanate, diisocyanate or triisocyanate

Reaction of the inventive polyisobutyl-substituted cyclohexanols (I) with isocyanates of the formula R^{a}—N═C═O in which R^{a} is an alkyl or aryl radical leads to N-substituted carbamic esters. Of great interest is the reaction with diisocyanates or triisocyanates, in particular when the inventive cyclohexanol (I) is a bifunctional product, i.e. a compound in which the polyisobutyl radical R¹ bears at least 2 cyclohexanol groups (I.a). These compounds are, as already mentioned, obtainable by using, as an alkylating agent for hydroxybenzenes, an at least bifunctionally terminated polyisobutene, in particular a polyisobutene which has a halide functionality or an olefinic double bond at least two chain ends. The reaction of such at least bifunctional cyclohexanols with di- or triisocyanates leads to the formation of polyurethanes. Suitable reaction conditions for this reaction correspond to those which are known from the prior art for urethane or polyurethane preparation and are described, for example, in Organikum, 17th edition, VEB Verlag der Wissenschaften, Berlin, p. 429 ff.
(e) Reaction with a carbonic acid derivative or with saturated or aromatic dicarboxylic acids or derivatives thereof.

This reaction too is effected preferably with at least bifunctional cyclohexanols (I) as described under (d).

Suitable carbonic acid derivatives are in particular the diesters, in particular the esters of C₁-C₄-alkanols, the monoester monohalides such as chloroformic acid, or phosgene.

Suitable derivatives of saturated dicarboxylic acids or aromatic carboxylic acids correspond to those which are specified under (a). Examples of suitable saturated dicarboxylic acids are oxalic acid, malonic acid, succinic acid, adipic acid and the like. Examples of suitable aromatic dicarboxylic acids are phthalic acid, isophthalic acid and terephthalic acid.

The reaction of the inventive, in particular at least bifunctional cyclohexanols (I) with these acids or derivatives thereof is effected generally under reaction conditions as known from the prior art for (poly)condensations and are described, for example, in the literature specified under (a) on ester formation.
(f) Reaction with ammonia or amines NHR^{a}R^{b}, where R^{a} is C₁-C₂₄-alkyl and R^{b} is H or C₁-C₂₄-alkyl, and if appropriate further reaction of the amine formed

Polyisobutyl-substituted cyclohexanols may be converted using ammonia or using primary or secondary amines to the corresponding polyisobutyl-substituted cyclohexylamines. Suitable processes are described, for example, in DE 1543377 or NL 6401010, which are hereby fully incorporated by reference. The procedure is similar to that in the hydrogenation reaction, except that ammonia or the amines specified are of course used for this purpose. The amination can be carried out either in the presence or in the absence of hydrogen. Preference is given to the reaction in the presence of hydrogen in order to prevent dehydrogenation of the cyclohexanol to the phenol. The hydrogen pressure is preferably from 1 to 100 bar, more preferably from 5 to 50 bar and in particular from 10 to 40 bar. With regard to suitable solvents, catalysts, amounts of catalyst and reaction temperatures, reference is made to the remarks made for the hydrogenation. Ammonia or the amine is used in an amount of from 0.5 to 200 mol, more preferably from 1 to 100 mol and in particular from 3 to 50 mol, based on 1 mole of hydroxyl functions which are comprised in the cyclohexanol group (I.a) of the polyisobutyl-substituted cyclohexanol (I). Alternatively, the amination can be effected simultaneously with the hydrogenation of the hydroxybenzene (II); however, preference is given to the successive procedure.

The resulting polyisobutyl-substituted cyclohexylamines may, if desired, be subjected to further derivatizations.

For example, they may be reacted with an olefinically unsaturated mono- or dicarboxylic acid or a suitable derivative thereof, which are as defined in (a), to give the corresponding amide. This can then, if desired, be polymerized as described under (a). Alternatively, the amine may be condensed with the polymer of an olefinically unsaturated mono- or dicarboxylic acid or a derivative thereof to give the corresponding polymeric amide. The condensation is suitably effected under reaction conditions as known from the prior art for the preparation of amides (see, for example, Jerry March, Advanced Organic Chemistry, 3rd edition, John Wiley & Sons, p. 370 ff).

Moreover, the cyclohexylamines, as described under (c) may be reacted with an alkylene oxide to give the corresponding alkyleneoxy-substituted product.

In addition, the polyisobutyl-substituted cyclohexylamines, analogous to (d), may be reacted with mono-, di- or triisocyanates to give N-substituted urea derivatives. Suitable reaction conditions are described, for example, in Jerry March, Advanced Organic Chemistry, 3rd edition, John Wiley & Sons, p. 802 ff. Of particular interest is the reaction of bifunctional cyclohexylamines, i.e. those products whose polyisobutyl radical bears two cyclohexylamine groups and is obtainable by the reaction of bifunctional cyclohexanols with amines or ammonia, with di- or triisocyanates to give polycondensation products.

Furthermore, the polyisobutyl-substituted cyclohexylamines, analogously to (e), may be condensed with a carbonic acid derivative such as phosgene or chloroformic esters, or else with urethanes, for example, to give urea derivatives or to give carbamic esters or (poly)urethanes. Moreover, the cyclohexylamines may be condensed with di- or polycarboxylic acids to give the corresponding amides. Here too, the reaction of bifunctional cyclohexylamines is of particular interest.

When the cyclohexylamines are bifunctional cyclohexylamines, as are formed in the reaction of bifunctional cyclohexanols with amines or ammonia, and they are then reacted with an at least bifunctional derivatizing agent, for example with a saturated, unsaturated or aromatic di- or polycarboxylic acid or derivatives thereof, with a carbonic acid derivative or with a di- or triisocyanate, the condensation products may, when suitable reaction conditions are selected, be oligomers, polymers or crosslinked polymers. When the derivatizing agent comprises olefinically unsaturated double bonds, these may, if desired, be oligomerized or polymerized, which forms polymeric condensation products.

Finally, the present invention further provides for the use of the inventive polyisobutyl-substituted cyclohexanols (I) or the above-described functionalization products thereof for the surface modification of organic or inorganic material, in particular as a hydrophilizing agent, lipophilizing agent, corrosion inhibitor, friction reducer, emulsifier, dispersant, adhesion promoter, binder, wetting agent or wetting inhibitor. The selection of suitable cyclohexanols (I) or functionalization products is guided specifically by the particular intended use and application medium and can be determined in the individual case by those skilled in the art.

For the surface modification with the inventive cyclohexanols (I) or functionalization products, suitable organic materials are, for example, polymers, in particular polyolefins such as polyethylene, polypropylene, polyisobutene and polyisoprene, and polyaromatics such as polystyrene, and also copolymers and mixtures thereof, the polymers preferably being in the form of films or moldings; cellulose, for example in the form of paper or cardboard; textiles composed of natural or synthetic fibers; leather; wood; mineral oil products such as fuels or lubricants; and additives for such mineral oil products such as lubricity improvers and cold flow improvers. Suitable inorganic materials are, for example, inorganic pigments, metal, glass and basic inorganic materials such as cement, gypsum or calcium carbonate.

In the context of the present invention, surface modification shall be understood to mean the change in the interface properties of the media admixed with the inventive cyclohexanols (I) or functionalization products. In this context, interfaces (phase interfaces) are understood to be surfaces which separate two immiscible phases from one another (gas-liquid, gas-solid, solid-liquid, liquid-liquid, solid-solid). These include the adhesion, sticking or sealing action, the flexibility, scratch or breakage resistance, wettability and wetting capability, sliding properties, frictional force, corrodibility, dyeability, printability and gas permeability of the application media. Accordingly, the inventive cyclohexanols (I) or functionalization products are preferably used as hydrophilizing agents, lipophilizing agents (hydrophobizing agents), corrosion inhibitors, friction reducers, emulsifiers, dispersants, adhesion promoters, binders, wetting agents, wetting inhibitors, volatizing agents or printing ink additives.

Preference is given to using the inventive cyclohexanols (I) and especially their functionalization products, in particular the polyacrylates, polyurethanes and polyesters, in paints, in particular in lacquers, and also in adhesives and sealants.

According to DIN 55945, paints are understood to be a liquid to pasty coating substance which is composed of binders, colorants (pigments or dyes), solvents or dispersants and also, if appropriate, fillers, siccatives, plasticizers and other additives. They serve to protect the particular substrate from moisture, soil, corrosion, fire, inter alia, but also to improve appearance. Paints are applied by brushing, rolling, spraying, dipping or casting and adapt in the liquid state to the surface of the substrate. After drying, a solid paint forms. The paints include, for example, lacquers and glazings. According to DIN 55945, lacquers are based on organic solvents. They are liquid or pulverulent-solid substances which are applied to objects in a thin layer and form an adhering solid film. Main components are binders, solvents (except in powdercoating materials), pigments (except in clearcoat materials), if appropriate fillers and coating assistants. Examples of lacquers are alkyd resin coatings, dispersion coatings, epoxy resin coatings, polyurethane coatings, acrylic resin coatings and cellulose nitrate coatings. Examples of glazings are wood protection glazings.

Telechelic cyclohexanols (I), i.e. those having at least 2 cyclohexanol groups (I.a), and also their reaction products with ammonia or primary/secondary amines are valuable macromers which can be used for the formation of networks (see, for example, Ivan, Kennedy, “Carbocationic Macromolecular Engineering”, Hanser Publishers 1992, pages 167 ff). The present invention therefore further provides for the use of polyisobutyl-substituted cyclohexanols of the formula (I) in which at least one of the R¹ radicals is substituted by at least one further cyclohexanol radical of the formula (I.a) as described above, or of corresponding inventive compositions which comprise such cyclohexanols, or of functionalization products thereof with ammonia or amines NHR^{a}R^{b}, in which R^{a} is C₁-C₂₄-alkyl and R^{b} is H or C₁-C₂₄-alkyl for the formation of networks.

The present invention is illustrated by the nonrestrictive examples which follow.

### EXAMPLES

### 1. Preparation of Polyisobutyl-Substituted Cyclohexanols (I)

1.1

1100 g of a 4-polyisobutylphenol which had been prepared from a polyisobutene having a number-average molecular weight Mₙ of 1000 (Glissopal 1000) were dissolved in 500 ml of heptane. The reaction solution was admixed with 500 mg of sodium hydride and the reaction mixture was transferred into a 3 l stirred pressure autoclave. After addition of 50 g of Raney nickel, hydrogen was introduced up to a pressure of 150 bar. Subsequently, the mixture was stirred at 100° C. for 2 hours and then at 150° C. for 1 hour. After the decompression and cooling, the Raney nickel catalyst was filtered off and the solvent was removed on a rotary evaporator at 140° C. and 5 mbar. 1050 g of 4-polyisobutylcyclohexanol were obtained as a colorless, clear oil.

¹H NMR (500 MHz; CDCl₃): δ: 3.97 (CH—OH: cis-cyclohexanol: 30%); 3.47 (CH—OH: trans-cyclohexanol: 70%)

1.2

1100 g of a 4-polyisobutylphenol which had been prepared from a polyisobutene having a number-average molecular weight Mₙ of 250 (isobutene oligomer having an average of 18 carbon atoms) were dissolved in 500 ml of heptane. The solution was then admixed with 500 mg of sodium hydride and the reaction mixture was transferred into a 3 l stirred pressure autoclave. After addition of 50 g of Raney nickel, hydrogen was introduced up to a pressure of 150 bar. The reaction mixture was left at 100° C. for 5 hours. Subsequently, the Raney nickel catalyst was filtered off and the solvent was removed on a rotary evaporator at 140° C. and 5 mbar. 914 g of 4-polyisobutyl-cyclohexanol were obtained in the form of a colorless, clear oil having a slight terpene-like odor.

¹H NMR (500 MHz; CDCl₃) δ: 4.03 (CH—OH: cis-cyclohexanol; 42%); 3.52 (CH—OH; trans-cyclohexanol: 58%)

### 2. Functionalization Examples

2.1 Esterification of the Product from Example 1.1 with Acrylic Acid

In a 2 l four-neck flask with internal thermometer, water separator with reflux condenser and gas inlet tube, 110 g (100 mmol) of the cyclohexanol from example 1.1 were dissolved in 200 ml of cyclohexane and saturated with air at room temperature by means of the gas inlet tube. Subsequently, the airstream was reduced to approx. 1 bubble per 5 s and kept constant over the entire reaction. The solution was admixed with a spatula-tip of methyl-hydroquinone and 1.34 g (14 mmol) of methanesulfonic acid. Subsequently, 7.9 g (110 mmol) of acrylic acid were added dropwise at 70° C. within 20 minutes. The reaction mixture was heated at 90° C. and reacted at this temperature for approx. 18 h. The mixture was then washed once with 150 ml of 15% NaCl solution, once with 100 ml of 0.5 M NaOH solution and again with 150 ml of 15% NaCl solution. The organic phase was dried over MgSO₄. Concentration of the solution afforded 112 g of the esterification product as a colorless, viscous oil.

¹H NMR (500 MHz, CD₂Cl₂): cis:trans ratio: 30:70;

cis product: 6.35 (dd, 1H); 6.12 (dd, 1H); 5.79 (dd, 1H); 5.04 (m, 1H); 1.95 (m, 2H);

trans product: 6.33 (dd, 1H); 6.08 (dd, 1H); 5.77 (dd, 1H); 4.67 (m, 1H); 2.03 (m, 2H); 1.84 (m, 2H).

For comparison:

cis reactant: 3.97 (m, 1H); 1.80 (m, 2H);

trans reactant: 3.47 (m, 1H); 1.96 (m, 2H); 1.78 (m, 2H).

1. A process for preparing polyisobutyl-substituted cyclohexanols of the formula (I), 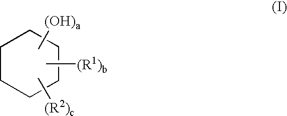
where
each R¹ is a polyisobutyl radical;
each R² is independently C₁-C₂₄-alkyl or C₁-C₂₄-alkoxy;
a and b are each independently from 1 to 3; and
c is from 0 to 4;
where the sum of a, b and c is from 2 to 6 and where each OH, R¹ and R² radical is bonded to different carbon atoms of the cyclohexane ring,
in which a polyisobutyl-substituted hydroxybenzenes of the formula (II) 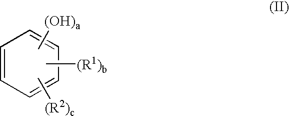
where R¹, R², a, b and c are each as defined above
is hydrogenated in the presence of a hydrogenation catalyst.

2. The process according to claim 1, wherein the polyisobutyl-substituted hydroxybenzene of the formula (II) is at least partly deprotonated before the hydrogenation.

3. The process according to claim 2, wherein deprotonation is effected with an alkali metal- or alkaline earth metal-containing inorganic base.

4. The process according to either or claims 2 and 3, wherein at least 0.1 mol % of the polyisobutyl-substituted hydroxybenzene of the formula (II) used is deprotonated.

5. The process according to any of the preceding claims, wherein hydrogenation is effected repeatedly.

6. The process according to any of the preceding claims, wherein the hydrogenation catalyst comprises at least one metal of transition group VIII.

7. The process according to claim 6, wherein the hydrogenation catalyst comprises nickel.

8. The process according to any of the preceding claims, wherein the hydrogenation is carried out in an alkane or alkane mixture as a solvent.

9. The process according to any of the preceding claims, wherein R¹ has a number-average molecular weight Mₙ of from 150 to 30 000.

10. The process according to any of the preceding claims, wherein R¹ is a radical derived from a reactive polyisobutene.

11. The process according to any of the preceding claims, wherein at least one R¹ radical in the hydroxybenzene of the formula (II) is substituted by at least one further hydroxybenzene radical (II.a) 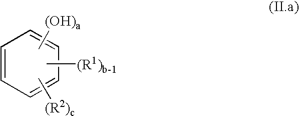
and wherein at least one R¹ radical in the cyclohexanol of the formula (I) is substituted by at least one further cyclohexanol radical (I.a) 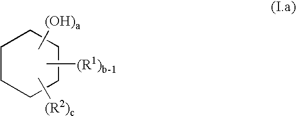
where R¹, R², a, b and c are each as defined in any of the preceding claims.

12. The process according to any of the preceding claims, wherein a and b in the formulae (I) and (II) are each 1.

13. The process according to any of the preceding claims, wherein c in the formulae (I) and (II) is 0.

14. A composition comprising polyisobutyl-substituted cyclohexanols of the formula (I) as defined in any of claims 1 or 9 to 13, obtainable by the process according to any of claims 1 to 13.

15. The composition according to claim 14, wherein R¹ in the polyisobutyl-substituted cyclohexanols of the formula (I) has a number-average molecular weight Mₙ of from 150 to 30 000.

16. A functionalization product of polyisobutyl-substituted cyclohexanols of the formula (I) as defined in any of claims 1 or 9 to 13, obtainable by reacting polyisobutyl-substituted cyclohexanols (I)
(a) with an olefinically unsaturated mono- or dicarboxylic acid or a derivative thereof and, if appropriate, subsequently polymerizing the olefinically unsaturated product formed, or with the polymer of an olefinically unsaturated mono- or dicarboxylic acid or a derivative thereof;
(b) with an allyl halide and, if appropriate, subsequently polymerizing the allyl ether formed;
(c) with an alkylene oxide;
(d) with an isocyanate, diisocyanate or triisocyanate;
(e) with a carbonic acid derivative or with saturated or aromatic dicarboxylic acids or derivatives thereof; or
(f) with ammonia or amines NHR^{a}R^{b}, where R^{a} is C₁-C₂₄-alkyl and R^{b} is H or C₁-C₂₄-alkyl, and if appropriate further reaction of the resulting amination product
(f.1) with at least one olefinically unsaturated mono- or dicarboxylic acid or a derivative thereof and, if appropriate, subsequently polymerizing the olefinically unsaturated product formed, or with the polymer of an olefinically unsaturated mono- or dicarboxylic acid or a derivative thereof;
(f.2) with an alkylene oxide; or
(f.3) with an isocyanate, diisocyanate or triisocyanate; or
(f.4) with a carbonic acid derivative or with saturated or aromatic dicarboxylic acids or derivatives thereof.

17. The use of polyisobutyl-substituted cyclohexanols of the formula (I) according to any of claims 1 or 9 to 13, or of compositions according to either of claims 14 and 15 or or functionalization products thereof according to claim 16 for the surface modification of organic or inorganic material.

18. The use of polyisobutyl-substituted cyclohexanols of the formula (I) as defined in any of claims 1 or 9 to 13 or of compositions according to either of claims 14 and 15 or of functionalization products thereof as defined in claim 16 in paints, lacquers, sealants and adhesives.

19. The use of polyisobutyl-substituted cyclohexanols of the formula (I) where at least one of the R¹ radicals is substituted by at least one further cyclohexanol radical of the formula (I.a) as defined in claim 11, or of functionalization products thereof with ammonia of amines NHR^{a}R^{b}, where R^{a} is C₁-C₂₄-alkyl and R^{b} is H or C₁-C₂₄-alkyl, for the formation of networks.
